(19) United States
(12) Patent Application Publication
(10) Pub. No.: US 2014/0088107 A1
(43) Pub. Date: Mar. 27, 2014

(54) OPHTHALMIC PREPARATION COMPRISING A PGF2ALPHA ANALOGUE

(71) Applicant: RATIOPHARM GMBH, Ulm (DE)

(72) Inventors: Dieter Swatscheck, Blaubeuren (DE); Max-Werner Scheiwe, Maulburg (DE); Michael Florenski, Ulm (DE)

(73) Assignee: RATIOPHARM GMBH, Ulm (DE)

(21) Appl. No.: 14/089,473

(22) Filed: Nov. 25, 2013

Related U.S. Application Data
(63) Continuation of application No. PCT/EP2012/059831, filed on May 25, 2012.

(30) Foreign Application Priority Data
May 27, 2011 (EP) 11167894.2

(51) Int. Cl. A61K 47/32; A61K 45/06; A61K 31/5575

(52) U.S. Cl. CPC A61K 47/32; A61K 31/5575; A61K 45/06
USPC 514/236.2; 514/573; 514/622; 514/530; 514/312

(57) ABSTRACT

The present invention relates to an aqueous ophthalmic preparation comprising a PGF2α analogue and at least one polyvinyl alcohol and the use thereof for the treatment of glaucoma and ocular hypertension.

### CROSS REFERENCE TO RELATED APPLICATION

This application is a continuation of international patent application PCT/EP2012/059831, filed May 25, 2012 designating the U.S., which international patent application was published in English and claims priority to European patent application EP 11 167 894.2, filed on May 27, 2011. The entire contents of these priority applications are incorporated herein by reference.

The present invention relates to ophthalmic preparations comprising a PGF2α analogue and uses thereof for the treatment of conditions of the eye.

Ophthalmic preparations comprising a PGF2α analogue per se are known and are commercially available, for example under the trade names “LUMIGAN” (Allergen, active ingredient Bimatoprost) or “TRAVATAN” (Alcon, active ingredient Travoprost).

Prostaglandin F2α (hereinafter “PGF2α”) analogues have been proven to be highly efficient compounds for the treatment of glaucoma and ocular hypertension. Their efficiency is such that they can be employed in ophthalmic compositions in very low concentrations. For example, LUMIGAN eye drops are available in two strengths containing either 0.01 or 0.03% (w/v) of Bimatoprost, and commercially available TRAVATAN eye drops contain 0.004% (w/v) of Travoprost. This low concentration of the active ingredient in the ophthalmological preparations in combination with the high lipophilicity of the active ingredient and resulting therefrom the high affinity to the polymer resins usually used for the containers for the ophthalmological preparations, poses a significant challenge when formulating stable preparations of such compounds.

These problems encountered when developing ophthalmological topical preparations comprising PGF2α analogues are compounded by the other requirements usually posed to ophthalmological preparations, in particular the need to ensure the long-term sterility of the preparation, in particular when packaged in a multi-use applicator and the need for a preparation with a low enough surface tension, so that a quick and efficient distribution of a topical preparation upon instillation into the eye can be ensured.

So far, in most ophthalmological topical preparations comprising PGF2α analogues these problems have been overcome by adding benzalkonium chlorides to the preparation. While initially these compounds were added to the preparations due to their antibacterial nature and primarily as preservatives, it has been shown that benzalkonium chlorides also have beneficial effects on the stability of preparations comprising PGF2α analogues and the surface tension of the resulting preparations. On the downside, preparations comprising benzalkonium chlorides tend to produce an unpleasant stinging or burning and sometimes even painful sensation in patients, when instilling the ophthalmological preparations into the eye, which negatively effects patient compliance. Furthermore, the use of preparations containing benzalkonium chlorides has led to corneal damage in some patients during long-term use, which is particularly worrying for preparations containing PGF2α analogues, as they tend to be used as long-term medications.

Due to the drawbacks of the use of benzalkonium chlorides, numerous attempts have been made to develop ophthalmic preparations comprising PGF2α analogues without the use of benzalkonium chlorides. One such product is sold under the trade name “TRAVATAN Z”, wherein benzalkonium chloride is replaced by a complex system comprising polyoxyl 40 hydrogenated castor oil, boric acid, propylene glycol, sorbitol and zinc chloride. A further preparation that is marketed as being preservative-free is sold in parts of Europe under the trade name “TAFLOTAN sine” and comprises disodium EDTA, glycerol and Polysorbate 80 in place of benzalkonium chloride.

Despite the fact that both “preservative-free” preparations do no longer contain benzalkonium chlorides, they now contain several other compounds instead, and, in particular, in both cases surfactants. The presence of multiple compounds obviously increases the chances of sensitization of a patient to one or several of the ingredients, as well as making the production thereof more expensive. Furthermore, the presence of surfactants might still lead to problems during long-term use. Finally, TAFLOTAN sine, at the moment, is only sold in single-dose containers indicating that there is a problem with the long-term sterility of the formulation if used in a multi-application container, which, in view of manufacturing costs, but also with regard to the waste generated, would be a lot more desirable.

It is, therefore, one object of the present invention to describe an aqueous ophthalmic preparation comprising a PGF2α analogue, whereby the preparation is essentially preservative-free, easy to manufacture and shows a long-term stability, both in view of the stability of the PGF2α analogue in solution and the long-term sterility of the preparation.

The inventors have now surprisingly found that by using polyvinyl alcohol, an aqueous ophthalmic preparation comprising a PGF2α analogue can be obtained that is storage-stable over a long period of time, shows the desired surface tension and can retain the sterility over a sufficiently long time, so that the preparation is suitable for use with multi dose containers for preservative eye drops. Furthermore, it has been shown that such a preparation can be formulated such that apart from the active ingredients and the at least one polyvinyl alcohol, only salts and buffers to adjust the pH and the tonicity of the solution need to be added, making the preparation inexpensive and simple to produce. It has surprisingly been found that by using polyvinyl alcohol aqueous ophthalmic preparations can be obtained that have a low enough surface tension to ensure that the preparation quickly disperses over the surface of the eye as well as a good penetration/ocular absorption of the active ingredient to ensure that the PGF2α analogue is quickly absorbed by the eye. Therefore such preparations can ensure the efficient treatment of e.g. glaucoma or ocular hypertension. Finally, polyvinyl alcohol has long been known as an ophthalmologically harmless excipient that can be used over extended periods of time without causing damage to a patient's eyes. On the contrary, polyvinyl alcohol is a common ingredient in artificial tears, so it is to be expected that the preparations of the present invention have the further advantage of a lubricating action in addition to the pharmacological action of the active ingredients.

The present invention relates to an aqueous ophthalmic preparation comprising a PGF2α analogue and at least one polyvinyl alcohol, whereby the solution is essentially preservative-free.

The invention further relates to the use of such a preparation in a method of treating a condition selected from the group consisting of glaucoma and ocular hypertension. The invention further relates to the use of an aqueous ophthalmic preparation of the invention for the manufacture of a medicament for the treatment of a condition selected from the group consisting of glaucoma and hypertension.

The invention further relates to a method of treating a condition selected from the group consisting of glaucoma and ocular hypertension in humans and animals, comprising administering an aqueous ophthalmic preparation according to the invention to the human or animal in need thereof.

The expression “PGF2α analogue” for the purpose of the invention relates to all PGF2α analogues by way of example and, preferably, to Bimatoprost, Latanoprost, Travoprost, Unoprostrone isopropyl and Tafluprost, as well as salts, solvates, complexes, prodrugs, or other pharmaceutically acceptable forms thereof.

The expression “essentially preservative-free” for the purpose of the present invention means that the preparation is completely free of preservatives or contains preservatives in amounts that are either not detectable or have no preservative effect.

In addition to the above ingredients the aqueous ophthalmic preparation of the invention can contain any further auxiliaries know to a person skilled in the art as long as they are not preservatives. Examples for such auxiliaries include auxiliaries to adjust the tonicity of the preparation such as sugars, e.g. dextrose, sugar alcohols, e.g. mannitol, alkali metal and alkaline earth metal halides, e.g. sodium or potassium chloride, alkali metal and alkaline earth metal nitrates, e.g. sodium or potassium nitrate and glycerol, buffering agents such as acetate, borate, citrate and phosphate buffers, viscosity modifiers such as cellulose and cellulose derivatives, e.g. methylcellulose or hydroxypropyl methylcellulose, hyaluronic acid and salts thereof, e.g. sodium hyaluronate and polyvinylpyrrolidone and antioxidants such as ascorbic acid and sodium tetrahydrogensulfite.

In an embodiment of the present invention, the PGF2α analogue is selected from the group consisting of Bimatoprost, Latanoprost, Travoprost, Unoprostone isopropyl and Tafluprost.

These PGF2α analogues are commercially available on the market and have been shown to be effective compounds for the treatment of conditions such as glaucoma and ocular hypertension.

In an embodiment of the invention, the preparation contains 0.001 to 0.05% (w/v), preferably 0.01 to 0.03% (w/v) of the PGF2α analogue.

The above-mentioned ranges have been shown to provide both, a safe and efficient treatment in the topical application of PGF2α analogues.

In an embodiment of the invention, the preparation contains 0.01 to 1.5% (w/v), preferably 0.02 to 1.0% (w/v), preferably 0.02 to 0.5% (w/v), in particular 0.05 to 0.3% (w/v) and especially 0.1 to 0.3% (w/v) of the at least one polyvinyl alcohol.

The above-mentioned ranges for the polyvinyl alcohol have been shown to be particularly advantageous, as below that range the beneficial effects of the addition of polyvinyl alcohol are lost, whereas the further addition of polyvinyl alcohol does not seem to have an additional beneficial effect and only raises the costs.

Furthermore, it is feared that too high a concentration of polyvinyl alcohol might lead to interactions with the active ingredients and, thereby, a reduction in the efficiency of the preparation.

In an embodiment of the invention, the polyvinyl alcohol is selected and added in an amount such that the preparation has a surface tension of 55 to 30 mN/m, preferably 50 to 35 mN/m and in particular 50 to 40 mN/m.

Preparations having a surface tension in the above mentioned ranges have the advantage that they quickly disperse in the eye therefore assisting the efficacy of the preparation.

The type and amount of the polyvinyl alcohol used can thereby be determined by a person skilled in the art without undue burden using standard experimental techniques. Examples of polyvinyl alcohols suitable thereby include those commercially available under the designation Mowiol and in particular Mowiol 4-88, Mowiol 8-88 and Mowiol 18-88.

In an embodiment of the invention, the preparation contains at least one further ingredient, preferably an active ingredient selected from the group consisting of the β-adrenergic receptor antagonists and, in particular, from the group consisting of Timolol, Propranolol and Carteolol.

It has been shown that combinations of PGF2α analogues with other active ingredients, in particular β-adrenergic receptor antagonists (beta blockers), and especially those already in use for ophthalmological applications such as Timolol, Propranolol and Carteolol, can increase the efficacy of the ophthalmic preparation.

In an embodiment of the invention, the ophthalmic preparation is essentially surfactant-fee.

The expression “essentially surfactant-free” for the purpose of the present invention means that the preparations contain no surfactant or amounts of surfactant which are either undetectable or have no effect as a surfactant. The term “surfactant” thereby includes all known anionic, cationic or nonionic surfactants. It goes without saying that polyvinyl alcohol is not considered a surfactant in the context of the invention.

A surfactant-free preparation has the general advantage over surfactant-containing preparations and they tend to be better tolerated by patients.

In a further embodiment, the ophthalmic preparation has essentially the following composition:

a) 0.01-0.03% (w/v) of Bimatoprost,

b) 0.0-1.0% (w/v) of Timolol maleate,

c) 0.01-0.05% (w/v) of citric acid,

d) 0.1-0.5% (w/v) of sodium monohydrogen phosphate,

e) 0.5-1.0% (w/v) of sodium chloride,

f) 0.05-0.15% (w/v) of polyvinyl alcohol, and

g) water.

For the purpose of the present invention, the expression “having essentially the following composition” means that the composition either comprises no other ingredients or, if further ingredients are present, they are not detectable or present in such amounts that they have no effect on the preparation as a whole.

It will be understood that the features of the invention mentioned above and those yet to be explained below can be used not only in the respective combination indicated, but also in other combinations or in isolation, without leaving the scope of the present invention.

The present invention is now further illustrated with the aid of the following non-limiting examples and with reference to the attached drawing in which,

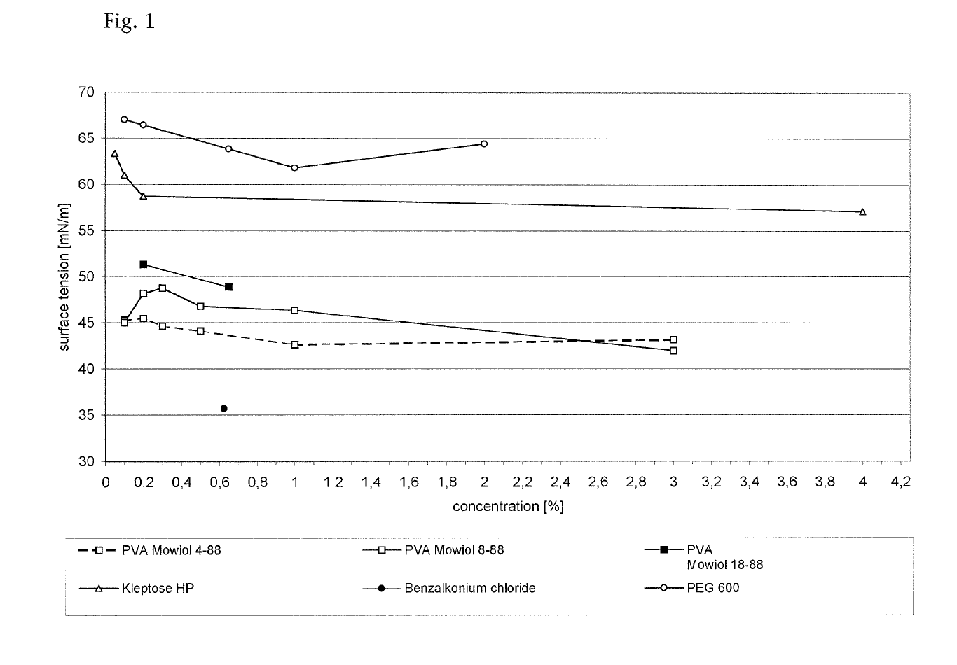

FIG. 1: shows a graph in which the surface tensions of exemplary compositions comprising different excipients are plotted against the concentration of the given excipient.

### EXAMPLES

### A.) Surface Tension

In order to investigate the influence of the excipients on the surface tension of the preparation solutions containing polyvinyl alcohol Mowiol 4-88, Mowiol 8-88 and Mowiol 18-88 in different concentrations were prepared and the surface tension of the solutions obtained was measured using a Tensiometer K12 (Krüss, Germany). The physicochemical data for the polyvinyl alcohols used are shown below in Table 1. The values obtained were compared with those of solutions comprising cyclodextrin Kleptose HP and PEG 600 in various combinations. The values for the surface tension for all solutions were plotted against the concentration of the respective excipient and the resulting graph is shown in FIG. 1. For reference purposes the surface tension of a composition containing 0.625% (w/v) of benzalkonium chloride is also included in FIG. 1.

The solutions obtained using polyvinyl alcohols showed a notably lower surface tension than those obtained using other excipients demonstrating the superior effect of the addition of polyvinyl alcohol.

TABLE 1

| | Mowiol 4-88 | Mowiol 8-88 | Mowiol 18-88 |
|---|---|---|---|
| pH (% in | 5.0-6.5 (4%) | 5.0-6.5 (4%) | 5.0-6.5 (4%) |
| water) | | | |
| Density | ca. 1.3 g/cm³ | ca. 1.3 g/cm³ | ca. 1.3 g/cm³ |
| Bulk density | 400-600 kg/m³ | 400-600 kg/m³ | 400-600 kg/m³ |
| Solubility in | insoluble | insoluble | insoluble |
| water at 20° C. | | | |
| Melting point | >200° C. | >200° C. | >200° C. |
| Viscosity | 3.4-4.6 mPas | 5.8-9.2 mPas | 15.3-20.7 mPas |
| (4%; water) | | | |
| Reference | Ph Eur, USP, JPE | Ph Eur, USP, JPE | Ph Eur, USP, JPE |
| CAS.Nr. | 25213-24-5 | 25213-24-5 | 25213-24-5 |
| Degree of | 85-89% | 85-89% | 85-89% |
| hydrolysis | | | |
| Molecular | 31.000 g/mol | 67.000 g/mol | 130.000 g/mol |
| weight | | | |
| Degree of | 630 | 1400 | 2700 |
| polymerisation | | | |

### B.) Exemplary Compositions

Using the ingredients stated in Table 2 in the given amounts, three exemplary ophthalmic preparations were produced.

TABLE 2

| | Preparation 1 | Preparation 2 | Preparation 3 |
|---|---|---|---|
| | 1000 ml | 1000 ml | 1000 ml |
| Bimatoprost | 0.30 g | 0.30 g | — |
| Travoprost | — | — | 0.04 g |
| Timolol maleat | 6.83 g | 6.83 g | 6.83 g |
| Glycerol 85% | — | — | 5.00 g |
| Citric acid monohydrate | 0.14 g | 0.14 g | |
| Disodium EDTA | — | — | 0.10 g |
| Sodium dihydrogen phosphate | 2.68 g | 2.68 g | |
| Sodium chloride | 6.80 g | 6.80 g | |
| Mannitol | — | — | 39.00 g |
| NaOH(A) | 45 gtt. | 94 gtt. | |
| Trometamol | — | — | 5.30 g |
| HCl 25% | — | — | q.s. |
| Polyvinyl alcohol | 0.50 g | 1.00 g | 3.00 g |
| (Mowiol 4-88) | | | |

The surface tension of preparation 1 and 2 was measured using a Tensiometer K12 (Krüss, Germany). Preparation 1 thereby had a surface tension of 45.23 mN/m and preparation 2 had a surface tension of 45.08 mN/m, which is slightly higher than a corresponding preparation comprising benzalkonium chloride for which a surface tension of 36.43 was measured, but still considered acceptable.

### C) Stability Studies

Preparations 1 and 2 were then subjected to accelerated stability tests under the conditions and with the results given below in table 3 and 4:

### Abbreviations:

DL: detection limit
QL: quantification limit
NP 1: Bimatoprost free acid
NP 2: by-product 1 of the Bimatoprost synthesis
NP 3: by-product 2 of the Bimatoprost synthesis
NP 4: (2RS)—N-(1,1-dimethylethyl)-2,3-bis[[4-(morpholin-4-yl)-1,2,5-thiadiazol-3-yl]-oxy]propan-1-amine
NP 5: 4-(Morpholin-4-yl)-1,2,5-thiadiazol-3-ol
NP 6: (2Z)-4-[(1S)-1-[[(1,1-dimethylethyl)amino]methyl]-2-[[4-(morpholin-4-yl)-1,2,5-thiadiazol-3-yl]oxy]ethoxy-4-oxobut-2-enoic acid
NP 7: 4-(4-chloro-1,2,5thiadiazazol-3-yl)morpholine

For NP 4 to NP 7 see also the impurities section in the Timolol maleate entry in the European Pharmacopeia 7.0.

TABLE 3
Stability studies for preparation 1

| | | Storage | | Storage | | Storage | |
|---|---|---|---|---|---|---|---|
| | | conditions: | | conditions: | | conditions: | |
| | | 4° C. | | 25° C./60% r.H. | | 40° C./75% r.H. | |
| Preparation 1 | Specification | 0 months | 1.5 months | 0 months | 1.5 months | 0 months | 1.5 months |
| Content/1 ml solution | | | | | | | |
| (HPLC) | | | | | | | |
| Bimatoprost | 95.0-105.0% | 102.73% | 103.76% | 102.73% | 103.65% | 102.73% | 102.61% |
| Content/1 ml solution | | | | | | | |
| (HPLC) | | | | | | | |
| Timolol maleat | 95.0-105.0% | 101.14% | 100.07% | 101.14% | 100.77% | 101.14% | 98.20% |
| Purity Test Bimatoprost | | | | | | | |
| Free acid (NP 1) | ≦0.2% | <DL | <DL | <DL | <DL | <DL | <DL |
| NP 2 | ≦0.2% | <DL | <DL | <DL | <DL | <DL | <DL |
| NP 3 | ≦0.2% | <DL | <DL | <DL | <DL | <DL | <DL |
| Purity Test Timolol | | | | | | | |
| NP 4 | ≦0.2% | <DL | <DL | <DL | <DL | <DL | <DL |
| NP 7 | ≦0.2% | <DL | <DL | <DL | <DL | <DL | <QL |
| NP 6 | ≦0.2% | <DL | <DL | <DL | <DL | <DL | <DL |
| NP 5 | ≦0.2% | <DL | <DL | <DL | <DL | <DL | <DL |

TABLE 4
Stability studies for preparation 2

| | | Storage | | Storage | | Storage | |
|---|---|---|---|---|---|---|---|
| | | conditions: | | conditions: | | conditions: | |
| | | 4° C. | | 25° C./60% r.H. | | 40° C./75% r.H. | |
| Preparation 2 | Specification | 0 months | 1.5 months | 0 months | 1.5 months | 0 months | 1.5 months |
| Content/1 ml solution | | | | | | | |
| (HPLC) | | | | | | | |
| Bimatoprost | 95.0-105.0% | 101.16% | 101.38% | 101.16% | 101.40% | 101.16% | 101.13% |
| Content/1 ml solution | | | | | | | |
| (HPLC) | | | | | | | |
| Timolol maleat | 95.0-105.0% | 97.50% | 101.39% | 97.50% | 100.54% | 97.50% | 100.11% |
| Purity Test Bimatoprost | | | | | | | |
| Free acid (NP 1) | ≦0.2% | <DL | <DL | <DL | <DL | <DL | <DL |
| NP 2 | ≦0.2% | <DL | <DL | <DL | <DL | <DL | <DL |
| NP 3 | ≦0.2% | <DL | <DL | <DL | <DL | <DL | <DL |
| Purity Test Timolol | | | | | | | |
| NP 4 | ≦0.2% | <DL | <DL | <DL | <DL | <DL | <DL |
| NP 7 | ≦0.2% | <DL | <DL | <DL | <QL | <DL | <QL |
| NP 6 | ≦0.2% | <DL | <DL | <DL | <DL | <DL | <DL |
| NP 5 | ≦0.2% | <DL | <QL | <DL | <DL | <DL | <DL |

From the results of the stability tests it becomes clear that the preparations of the invention show the desired stability with regard to both the content in Bimatoprost and Timolol as well as the formation of by-products.

1. An aqueous ophthalmic preparation comprising a PGF2α analogue and at least one polyvinyl alcohol, wherein the preparation is essentially preservative-free.

2. The aqueous ophthalmic preparation of claim 1, wherein said PGF2α analogue is selected from the group consisting of Bimatoprost, Latanoprost, Travoprost, Unoprostone isopropyl and Tafluprost.

3. The aqueous ophthalmic preparation of claim 1, comprising 0.001 to 0.05% (w/v), of said PGF2α analogue.

4. The aqueous ophthalmic preparation of claim 1, comprising 0.01 to 0.03% (w/v) of said PGF2α analogue.

5. The aqueous ophthalmic preparation of claim 1, comprising 0.01 to 1.5% (w/v) of said at least one polyvinyl alcohol.

6. The aqueous ophthalmic preparation of claim 5, comprising 0.02 to 1.0% (w/v) of said at least one polyvinyl alcohol.

7. The aqueous ophthalmic preparation of claim 6, comprising 0.02 to 0.5% (w/v) of said at least one polyvinyl alcohol.

8. The aqueous ophthalmic preparation of claim 7, comprising 0.05 to 0.3% (w/v) of said at least one polyvinyl alcohol.

9. The aqueous ophthalmic preparation of claim 1, comprising 0.1 to 0.3% (w/v) of said at least one polyvinyl alcohol.

10. The aqueous ophthalmic preparation of claim 1, when said polyvinyl alcohol is selected and added in an amount such that the preparation has a surface tension of 55 to 30 mN/m.

11. The aqueous ophthalmic preparation of claim 1, when said polyvinyl alcohol is selected and added in an amount such that said preparation has a surface tension of 50 to 35 mN/m.

12. The aqueous ophthalmic preparation of claim 1, when said polyvinyl alcohol is selected and added in an amount such that said preparation has a surface tension of 50 to 40 mN/m.

13. The aqueous ophthalmic preparation of claim 1, comprising at least one further active ingredient.

14. The aqueous ophthalmic preparation of claim 13, wherein said at least one further active ingredient is selected from the group consisting of the β-adrenergic receptor antagonists Timolol, Propranolol and Carteolol.

15. The aqueous ophthalmic preparation of claim 1, wherein it is essentially surfactant free.

16. The aqueous ophthalmic preparation of claim 1, having essentially the following composition:
a.) 0.01-0.03% (w/v) of Bimatoprost,
b.) 0.0-1.0% (w/v) of Timolol maleate,
c.) 0.01-0.05% (w/v) of citric acid,
d.) 0.1-0.5% (w/v) of sodium monohydrogen phosphate,
e.) 0.5-1.0% (w/v) of sodium chloride,
f.) 0.05-0.3% (w/v) of polyvinyl alcohol, and
g.) water.

17. The aqueous ophthalmic preparation of claim 1 for the treatment of a condition selected from the group consisting of glaucoma and ocular hypertension.

18. A method of treating a condition selected from the group consisting of glaucoma and ocular hypertension in humans and animals comprising administering an aqueous ophthalmic preparation comprising a PGF2α analogue and at least one polyvinyl alcohol, whereby the preparation is essentially preservative-free to a human or animal in need thereof.
